(19) United States
(12) Patent Application Publication
(10) Pub. No.: US 2009/0069547 A1
(43) Pub. Date: Mar. 12, 2009

(54) STABILIZATION OF MAMMALIAN MEMBRANE PROTEINS BY SHORT SURFACTANT PEPTIDES

(75) Inventor: SHUGUANG ZHANG, Lexington, MA (US)

Correspondence Address: ELMORE PATENT LAW GROUP, PC, 515 Groton Road, Unit 1R, Westford, MA, 01886, US

(73) Assignee: Massachusetts Institute of Technology, Cambridge, MA (US)

(21) Appl. No.: 12/113,365

(22) Filed: May 1, 2008

Related U.S. Application Data
(60) Provisional application No. 60/915,132, filed on May 1, 2007.

(51) Int. Cl. C07K 1/00

(52) U.S. Cl. 530/402

(57) ABSTRACT

The present invention provides for a method and kits for stabilizing mammalian membrane protein using short surfactant peptides. The mammalian membrane protein can be a G protein-coupled receptor.

### RELATED APPLICATION

This application claims the benefit of U.S. Provisional Application No. 60/915,132, filed on May 1, 2007. The entire teaching of the above application is incorporated herein by reference.

### GOVERNMENT SUPPORT

The invention was supported, in part, by a grant NSF CCR-0122419 from National Science Foundation. The Government has certain rights in the invention.

### BACKGROUND OF THE INVENTION

Approximately one-third of the genes in the human genome encode membrane proteins. Membrane proteins play a role in many important cellular activities including energy conversion, cell signaling, cell-cell interactions, cell adhesion, cell migration, protein trafficking, viral fusion, neural synaptic activities and ion and metabolite transport. Membrane proteins are embedded in the lipid bilayer of the cell membrane and are comprised of both hydrophobic and hydrophilic moieties. A significant portion of membrane proteins are G-protein coupled receptors (GPCRs). GPCRs are a superfamily of transmembrane proteins characterized by seven membrane spanning domains. These proteins are vitally important in the cellular signal transduction by transducing an extracellular signal (such as ligand-receptor binding) into an intracellular signal or activation of G-protein.

Although membrane proteins like GPCRs are major targets of drug discovery efforts, very little is known about the structure of these proteins. Because membrane proteins possess both hydrophobic and hydrophilic regions, they are difficult to solubilize, extract and purify. One of the challenges posed by membrane proteins is that they are subject to rapid denaturation and/or aggregation in solution. Despite the availability of a wide range of surfactants, few provide increased and/or prolonged stability of membrane proteins in solution. Therefore, there remains a need in the art for surfactants capable of increasing the stability of membrane proteins such as G protein-coupled receptors.

### SUMMARY OF THE INVENTION

The present invention provides for a method of stabilizing a mammalian membrane protein using short surfactant peptides. In a further embodiment, the invention is a method of stabilizing a mammalian membrane protein in its native state using short surfactant peptides. In another embodiment, the invention is directed to a kit for stabilizing a mammalian membrane protein including a composition comprising a short surfactant peptide and a composition comprising a mammalian membrane protein. In a further embodiment, the mammalian membrane protein is a mammalian G protein-coupled receptor.

In one embodiment, one or more surfactant peptides are used to stabilize the mammalian protein. In a further embodiment, the method comprises the addition of a non-peptide surfactant in addition to the peptide surfactant.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a proposed model of rhodopsin stabilization using peptide surfactants. GPCR bovine rhodopsin was extracted with OG from the membrane. After surfactant exchanges, the hydrophobic alanine tail of A₆D forms the rhodopsin-surfactant complex only on the belt area. Small peptide surfactants surround rhodopsin and act to protect it from thermal denaturation, similar to the action of chaperones. This action may be similar to that of lipids and other surfactants. The proposed dimeric GPCR bovine rhodopsin is embedded in the cellular membrane. The lipids of the membrane form bilayers. The surfactants are OG, which is used for the initial purification, and peptide surfactant A6D. The lipids have two tails, and both OG and A6D have a single tail.

FIGS. 2A-2D show UV-visible absorption spectra of GPCR bovine rhodopsin (Rho) in PBS containing 1% OG (A), 0.9% PEG/1% OG (B), 2.5 mM A6D/1% OG (C), or 2.8 mM RAD8/1% OG (D). Spectra were recorded every 5 min, up to 120 min, at 40° C. “L” indicates lipid.

FIGS. 3A-3C shows stability kinetics of rhodopsin (Rho) in different surfactants. Thermal stability of rhodopsin was measured as a rate of the decay at A₅₀₀. The A₅₀₀ spectra at different time points are expressed as percentage of absorbance at the initial state of the experiment. (A) The half-life of rhodopsin in different surfactants (as described in the FIG. 2 legend) was as follows: 71 min in 1% OG, 90 min in 0.9% PEG/1% OG, 173 min in 2.5 mM A6D/1% OG, and 99 min in 2.8 mM RAD8/1% OG. (B) Stability of rhodopsin as a function of the concentration of the peptide surfactant A6D at 40° C. Half-life of rhodopsin was as follows: 277 min in 3.75 Mm A6D/1% OG, 173 min in 2.5 mM A6D/1% OG, 101 min in 1.25 mM A6D/1% OG, and 71 min in 1% OG (PBS). (C) Stability of rhodopsin in DM with or without A6D at 50° C. Half-life of rhodopsin was as follows: not available in 2.5 mM A6D/9.8 mM DM, 96 min in 19.6 mM DM, 96 min in 12.3 mM DM, and 102 min in 9.8 mM DM.

FIGS. 4A-4C shows kinetics of bovine rhodopsin under different conditions. (A) Stability of rhodopsin in the absence of OG at different temperatures. Half-life of rhodopsin was as follows: not available in 2.5 mM A₆D at 40° C., 50° C., and 55° C.; 101 min in control solution (1.25 mM A₆D/1% OG) at 40° C.; <5 min in control solution at 50° C. (B) Decay of A₅₀₀ in delipidated rhodopsin in the absence of OG. Half-life of rhodopsin was as follows: 122 min in 1.25 mM A₆D, 47 min in PBS, and 27 min in 1% OG (control). (C) Stability of delipidated rhodopsin at 40° C. Half-life of rhodopsin was as follows: 128 min in 3.75 mM A6D/1% OG, 76 min in 1.87 mM A6D/1% OG, 39 min in 0.9% PEG/1% OG, and 23 min in 1% OG.

### DETAILED DESCRIPTION OF THE INVENTION

The present invention is directed to methods and kits for stabilizing mammalian membrane proteins comprising adding a surfactant peptide to a mammalian membrane protein.

As used herein, “a” or “an” is meant to encompass one or more unless otherwise specified.

As used herein, the term “amino acid” encompasses a naturally or non-naturally occurring amino acid. Amino acids are represented by their well-known single-letter designations: A for alanine, C for cysteine, D for aspartic acid, E for glutamic acid, F for phenylalanine, G for glycine, H for histidine, I for isoleucine, K for lysine, L for leucine, M for methionine, N for asparagines, P for praline, Q for glutamine, R for arginine, S for serine, T for threonine, V for valine, W for tryptophan and Y for tyrosine.

The term “physiologic pH” is a pH of about 7.

The term “self-assembly” is a process of atoms, molecules or peptides forming regular shaped structures or aggregates in response to conditions in the environment, such as when added to an aqueous medium.

The term “critical aggregation concentration” or “CAC” is the concentration above which the surfactant peptides aggregate or form regular shaped structures, such as micelles, nanotubes or nanovessicles.

The term “non-peptide surfactant” encompasses surfactants other than the surfactant peptides of the present invention. A surfactant is a compound that is amphiphilic or that contains both hydrophobic groups (their “tails”) and hydrophilic groups (their “heads”). Surfactants are soluble in both organic solvents and water.

As used herein, the phrase “stabilization of a mammalian membrane protein” is meant to include stabilization of a mammalian membrane protein in its native form and remain functional.

The terms “peptide surfactant,” “surfactant peptide” and “peptergent” are used interchangeably herein.

A surfactant peptide is a peptide that has a sequence of less than or equal to 10 amino acids with a physical structure wherein the peptide surfactant has a hydrophilic head group and a lipophilic tail group. The hydrophilic head group is comprised of a polar and/or charged (either positively or negatively charged at physiological pH) amino acid. The hydrophobic head group is comprised of a hydrophobic amino acid such as a non-polar and/or uncharged amino acid. In one embodiment, the hydrophilic amino acid is positively charged at physiological pH. In another embodiment, the hydrophilic amino acid is negatively charged at physiological pH. When dissolved in water or in an ionic solution, the peptide surfactants undergo self-assembly to form micelles, nanovesicles or nanotubes. Surfactant peptides have been described, for example, in U.S. Pat. No. 7,179,784, U.S. Patent Application Publication 2003/0176335 A1 and U.S. Patent Application Publication No. 2006/0211615 A1, the teachings of which are incorporated by reference herein.

In one embodiment, the peptide surfactant has a formula selected from the group consisting of:

a. (Φ)ₘ(+)ₙ (Formula (I)),

b. (+)ₙ(Φ)ₘ (Formula (II)),

c. (Φ)ₘ(−)ₙ (Formula (III)),

d. (−)ₙ(Φ)ₘ (Formula (IV)),

e. (−)ₙ(Φ)ₘ(−)ₙ (Formula (V)),

f. (+)ₙ(Φ)ₘ(+)ₙ (Formula (VI)),

g. (Φ)ₘ(−)ₙ(Φ)ₘ (Formula (VII)),

h. (Φ)ₘ(+)ₙ(Φ)ₘ (Formula (VIII)),

i. (+)ₙ(Φ)ₘ(−)ₙ (Formula (IX)),

j. (−)ₙ(Φ)ₘ(+)ₙ (Formula (X)),

k. (τ)ₙ(Φ)ₘ (Formula (XI)), and

l. (Φ)ₘ(τ)ₙ (Formula (XII)),

m. (τ)ₙ(Φ)ₘ(τ)ₙ, (Formula (XIII)),

n. (Φ)ₘ(τ)ₙ(Φ)ₚ, (Formula (XIV)),

wherein:

each (Φ) is independently a non-polar amino acid containing a non-charged side chain at physiological pH;

each (+) is independently an amino acid containing a cationic side chain at physiological pH;

each (Φ) is independently an amino acid containing an anionic side chain at physiological pH;

each (τ) is independently is a polar amino acid containing a non-charged side chain at physiological pH, for example, serine, threonine, asparagine and glutamine;

each m is independently an integer greater than or equal to 4; and

each n is independently an integer greater than or equal to 1.

Reading each of the Formulae (I) to (XIV) from left to right corresponds to the amino acid sequence from the N-terminus to the C-terminus. In other words, the first amino acid in the sequence is the N-terminus and the last amino acid in the sequence is the C-terminus.

Each (Φ), m and n are as defined for Formulae (I) to (X) above. Reading each of the Formulae (XI) to (XII) from left to right corresponds to the amino acid sequence from the N-terminus to the C-terminus. Hydrophilic amino acids include, but are not limited to aspartic acid, glutamic acid, lysine, arginine, histidine, serine and threonine. In one embodiment, each (τ) is independently is a polar amino acid that is uncharged at physiological pH. In another embodiment, each (τ) is independently selected from the group consisting of serine and threonine. In another embodiment, the peptide surfactant is selected from the group consisting of SSSAAAA (S₃A₄) and SSSAAAAA (S₃A₅). S₅A₄-5 which is encompassed the Formulae XI and XII.

In a further embodiment, the surfactant peptide has a formula selected from the group consisting of (Φ)ₘ(+)ₙ (Formula (I)), (+)ₙ (Φ)ₘ (Formula (II)) (Φ)ₘ(−)ₙ (Formula (III)) and (−)ₙ(Φ)ₘ (Formula (IV)).

In one embodiment, the surfactant peptide has the formula (Φ)ₘ(+)ₙ. Surfactant peptides having the formula (Φ)ₘ(+)ₙ include, but are not limited to, AAAAAAK (A₆K), VVVVVVK (V₆K), IIIIK (I₄K), IIIIIIKK (I₆K₂), VVVVVVKK (V₆K₂) and VVVVVVRR (V₆R₂). Ac-GAVILK—NH₂, Ac-GAVILR—NH₂, RLIVAG-NH₂, KLIVAG-NH₂.

In another embodiment, the surfactant peptide has the formula (Φ)ₘ(−)ₙ. Surfactant peptides having the formula (Φ)ₘ(−)ₙ include, but are not limited to, AAAAAAD (A₆D), VVVVVVD (V₆D), VVVVVVDD (V₆D₂), LLLLLLD (L₆D), LLLLLLDD (L₆D₂), IIIIIID (I₆D), FFFFFFD (F₆D), GGGGGGD (G₆D), GGGGGGDD (G₆D₂) and GGGGGGGGDD (G₈D₂). Ac-GAVILD-NH₂, Ac-GAVILE-NH₂, DLIVAG-NH₂, ELIVAG-NH₂.

In yet another embodiment, the surfactant peptide has the formula (+)ₙ(Φ)ₘ. Surfactant peptides having the formula (+)ₙ(Φ)ₘ include, but are not limited to, KKLLLLLL (K₂L₆), KKVVVVVV (K₂V₆) and KAAAAAA (KA₆). Ac-GAVILK—NH₂, Ac-GAVILR—NH₂, RLIVAG-NH₂, KLIVAG-NH₂.

In a further embodiment, the surfactant peptide has the formula (−)ₙ(Φ)ₘ. Surfactant peptides having the formula (−)ₙ(Φ)ₘ include, but are not limited to, DIIII (DI₄), DGGGGGG (DG₆), DAAAAAA (DA₆), DVVVVVV (DV₆), DIIIIII (DI₆), DLLLLLL (DL₆) and DFFFFFF (DF₆). Ac-GAVILD-NH₂, Ac-GAVILE-NH₂, DLIVAG-NH₂, ELIVAG-NH₂.

In a further embodiment, the surfactant peptides as described above substitute a hydrophilic, non-charged polar group for the charged group. Examples include (τ)ₙ(Φ)ₘ (Formula (XI)) and (Φ)ₘ(τ)ₙ (Formula (XII)), wherein each (τ) is independently a hydrophilic amino acid, including polar amino acids containing a non-charged side chain at physiological pH, for example, serine, threonine, asparagine and glutamine. The terminal amino acids may each independently and optionally be blocked.

In one embodiment, each (Φ) is independently selected from the group consisting of alanine, valine, leucine, isoleucine, glycine, phenylalanine and proline. In another embodiment, each (+) is independently selected from the group consisting of lysine, arginine and histidine. In a further embodiment, each (−) is independently selected from the group consisting of aspartic acid and glutamic acid.

In each instance, where a pH other than physiological pH is to be used, the amino acids can be substituted with those that will be charged or not charged, or hydrophilic, as is necessary or desirable. Thus, the use of physiological pH in each instance where stated herein is to be understood as being a preferred but not necessary embodiment.

In one embodiment, the peptide surfactant has between 6 and 10 amino acids. In another embodiment, each n is independently an integer between 1 and 3. In an additional embodiment, each m is independently an integer greater than or equal to 5. In a further embodiment, each m is independently an integer between 5 and 9.

In a further embodiment, the N-terminus of the surfactant peptide is blocked, e.g., acylated or acetylated. In an additional embodiment, the C-terminus of the surfactant peptide is blocked, e.g., esterified or amidated. Blocking the N or C terminus can improve the surfactant properties by controlling the charge. For example, it may be preferred to block the terminus located at the hydrophobic tail. It may also be preferred to block the terminus at the hydrophilic tail where the terminal charge differs from the side chains. Non-limiting examples of acetylated surfactant peptides include ac-I₆K₂—CONH₂, ac-A₆K—CONH₂, ac-A₆D-COOH, ac-V₆K₂—CONH₂ and ac-V₆R₂—CONH₂), ac-A₆K—COOH, ac-I₆K₂—CONH₂, ac-KA₆-CONH₂ (KA₆-NH₂), DA₆-NH₂ and ac-V₆R₂—NH₂ (wherein “ac” represents an acetyl group). In another embodiment, the peptide surfactant has a length from about 2 to about 3 nm. In a further embodiment, the surfactant peptide forms a nanotube or a nanovessicle with a diameter between about 30 to about 50 nm. In an additional embodiment, the peptide surfactant has a critical aggregation concentration in a few μM to millimolar range.

The method of the invention comprises adding a surfactant peptide to a mammalian membrane protein to stabilize the mammalian membrane protein. The mammalian membrane protein is an isolated and/or purified mammalian membrane protein. The surfactant peptide is added to the mammalian membrane protein in an amount sufficient to increase the stability of the mammalian membrane protein. In one embodiment, the surfactant peptide is added to the mammalian membrane protein in an amount sufficient to increase the thermal stability of the mammalian membrane protein.

In one embodiment, the method of stabilizing a mammalian membrane protein comprises the following steps:
(a) isolating the mammalian membrane protein;
(b) adding the isolated membrane protein to an aqueous solution; and
(c) adding a peptide surfactant having a formula selected from Formulae (I) to (XII) to the aqueous solution.

In a second embodiment, the method comprises the following steps:
(a) isolating the mammalian membrane protein;
(b) adding the isolated membrane protein to an aqueous solution;
(c) adding a non-peptide surfactant to the aqueous solution; and
(d) adding a peptide surfactant having a formula selected from Formulae (I) to (XII) to the aqueous solution.

In a third embodiment, the method comprises the following steps:
(a) isolating the mammalian membrane protein and
(b) drying the mammalian membrane protein on a surface in the presence of a surfactant peptide having a formula selected from Formulae (I) to (XII).

The surface can be any surface that does not react with the mammalian membrane protein and/or the surfactant peptide. Exemplary surfaces include, but are not limited to, glass slides, plastic slides, a multi-well plate and the like. As used herein, drying is meant to include the removal of water or dehydration of the mammalian membrane protein. Drying can be accomplished by methods well known in the art such as lyophilization and/or flash freezing in liquid nitrogen.

In a further embodiment, the surfactant peptide is added to the mammalian membrane protein at a concentration that is based on the CAC of the surfactant peptide. The CAC of surfactants can be determined experimentally using known dynamic light scattering methods. A minimal amount of sample can be used in this method. Each CAC determination takes a few hours, therefore it is possible to determine the CAC for a large number of peptide detergents in a few weeks. It is known that the lower the CAC, the more hydrophobic the detergents and the stronger the aggregation in water. The CAC for several peptide surfactants in water is shown below in Table 1.

TABLE 1

| Peptide Surfactant | CAC |
|---|---|
| A₆D | ~1.6 mM |
| A6K | ~1.5 mM |
| V₆D | ~0.7 mM |
| V₆D₂ | 1.1 mM |
| pSA6 | 0.7 mM |
| pSV6 | 0.09 mM |

Wherein pS is phosphoserine.

In another embodiment, the surfactant peptide is added to the mammalian membrane protein at a concentration from about 1 times the CAC of the surfactant peptide (1×CAC) to a concentration that is about 30 times the CAC of the surfactant peptide (30×CAC). In one embodiment, the surfactant peptide is added to the mammalian membrane at a concentration which is at least 1.5 times the CAC of the surfactant peptide (1.5×CAC). In a second embodiment, the surfactant peptide is added at a concentration that is at least 2 times the CAC of the surfactant peptide (2×CAC). In a third embodiment, the surfactant peptide is added at a concentration that is at least 5 times the CAC of the surfactant peptide (5×CAC). In a fourth embodiment, the surfactant peptide is added at a concentration that is at least 10 times the CAC of the surfactant peptide (10×CAC). In a fifth embodiment, the surfactant peptide is added at a concentration that is at least 12 times the CAC of the surfactant peptide (12×CAC). In a sixth embodiment, the surfactant peptide is added at a concentration that is at least 15 times the CAC of the surfactant peptide (15×CAC). In a seventh embodiment, the surfactant peptide is added at a concentration that is at least 20 times the CAC of the surfactant peptide (20×CAC).

In another embodiment, the surfactant peptide is added to the mammalian membrane protein on or in a dry surface. In an additional embodiment, the surfactant peptide is added to the membrane protein in an aqueous medium. In one embodiment, the aqueous medium is an aqueous solution. In a further embodiment, the surfactant peptide is added to the membrane protein in an ionic solution.

In one embodiment, the method comprises the addition of a single type of surfactant peptide. In another embodiment, the method comprises the addition of at least two different surfactant peptides. The at least two different surfactant peptides can have the same or different formulae (referring to Formulae I to XII above). For example, the two different surfactant peptides can both be encompassed by the Formula (I). As another non-limiting example, one of the two different surfactant peptides can be encompassed by the Formula I and the other surfactant peptide can be encompassed by the Formula III.

In yet another embodiment, a surfactant peptide and a non-peptide surfactant are added to the mammalian membrane protein. The term “non-peptide surfactant” is meant to encompass surfactants other than the surfactant peptides of the present invention. A surfactant is a compound that is amphiphilic or that contains both hydrophobic groups (their “tails”) and hydrophilic groups (their “heads”). Surfactants are soluble in both organic solvents and water. There are generally two types of surfactants, ionic and non-ionic surfactants. Ionic surfactants are surfactants that have a net charge at their heads. Non-ionic surfactants are surfactants that have no net charge at their heads. Examples of non-peptide surfactants include, but are not limited to polyoxyalkylene sorbitan fatty acid esters, sorbitan fatty acid esters, alkylene glycol fatty acid esters, polyoxyalkylene fatty acid esters, fatty acid esters, polyoxyalkylene fatty acid ethers, C₁₆C₂₄ fatty acids, fatty acid mono-, di- or poly-glycerides, polyoxyalkylene alkyl phenols, alkyl phenyl ethers, polyoxyethylene polyoxypropylene block copolymers, fatty amine oxides, fatty acid alkanolamides, alkyl cellulose, carboxyalkyl cellulose and polyoxyalkylene castor oil derivatives. Ionic surfactants include, but are not limited to, alkyl sulfates, olefin sulfates, ether sulfates, monoglyceride sulfates, alkyl sulfonates, aryl sulfonates, olefin sulfonates, alkyl sulfosuccinates, aryl sulfosuccinates, including sodium lauryl sulfate, dioctyl sodium sulfosuccinate and dioctyl sodium sulfonate), benzalkonium salts, polyoxyalkylene alkylamines, alkylamines, alkanolamine fatty acid esters, quaternary ammonium fatty acid esters, dialkyl ammonium salts, alkyl pyridinium salts including stearylamine and triethanolamine oleate, benzethonium chloride. Non-limiting examples of non-peptide surfactant are lauryldimethyamine oxide (LDAO), n-dodecyldimethyamine N-oxide (NDAO), Octyldimethyamine N-oxide (ODAO), undecyldimethyamine N-oxide (UDAO), Octyl-β-D-glucose (β-OG), Decyl-β-D-glucose (β-DG), Nonyl-β-D-glucose (β-NG), Dodecyl-β-D-maltoside (DDM), Octyanoylsucrose (OS), Octyl-β-D-galactoside (β-OGal) and Dodecyl phosphocholine (DPC). In one embodiment, the non-peptide surfactant used in the method of the invention is a non-ionic surfactant. In a further embodiment, the non-ionic surfactant is selected from the group consisting of n-dodecyl-B-D-maltoside and octyl-D-glucoside. In yet another embodiment, the non-peptide surfactant is added in an amount between about 2 and about 200 times the CAC of the non-peptide surfactant.

Mammalian membrane proteins that can be stabilized using a method or kit of the present invention include, but are not limited to, rhodopsin, chemokine (C—C motif) receptor 3 (CCR3), chemokine (C—C motif) receptor 5, CD4 receptor, olfactory receptors (including, for example, mouse IG7 receptor (also known as mOR276-1), human 1D2 receptor (also known as hOR17-4)). In one embodiment, the mammalian membrane protein is a G protein-coupled receptor. In another embodiment, the mammalian membrane protein has a molecular weight from about 25,000 Daltons to about 500,000 Daltons. In a further embodiment, the mammalian membrane protein has a molecular weight over about 25,000 Daltons.

Preferably, the protein is selected from the group consisting of the following GPCR proteins TDAG8, LPA1, LPA2, LPA3, LPA4, ET receptors, GPR4, GPR54, CCK1, CCK2, GPRP, CXCR4, PAR1, EP2, EP4, CXCR2, GPR41, GPR43, Smoothened, NMB-R, VIPR1, VIPR2, LH receptor, ATI, B1, B2, Frizzleds, GPR30, ACTHR, TSH receptor, GPR105, β1AR, β2AR, CASR, MC1R, OGR1, CXCR1, CXCR2, CXCR3b, CXCR3, CXCR4, CXCR5, CXCR6, CCR2, CCR5, CCR1, CCR3, CCR4, CCR6, CCR7, CCR8, CCR9, CCR10, XCR1, CX 3 CR1, Y1, SCL-1, OX1R, OX2R, MC4R, Y4, Y5, Y1, Y2, Y5, GPR10, GPR39, GLP120, GPR40, GHS-R, H3R, PAC1-R, GPR41, GPR43, OX1, OX2, Adenosine A2A receptor, SCL-1, OX1, OX2, LPA1, P2Y2, P2Y4, P2Y6, GPR41, β2 adrenergic receptor, AdipoR1, AdipoR2, Histamine receptors H1, H2, and H3, APJ, CMKRL1, BLTR, G2A, ETAR, ETBR, PAR-2, ETAR, ETBR, LPA3, GPR135, PAR-3, and PAR-4.

In one embodiment, addition of a surfactant peptide provides at least about a 1.2-fold increase in stability compared to the stability of a mammalian membrane protein in the presence of a non-peptide surfactant. In another embodiment, addition of a surfactant peptide provides at least about 1.5, at least about 1.7, at least about 2.0, at least about 2.2, at least about 2.3 or at least about 2.5-fold increase in stability compared to the stability of a mammalian membrane protein in the presence of a non-peptide surfactant.

The present invention also encompasses kits for stabilizing mammalian membrane proteins. The kit comprises a composition comprising a mammalian membrane protein and a composition comprising a surfactant peptide having a formula selected from Formulae (I) to (XII). The kit may comprise one or more surfactant peptides. Kits according to the invention may further comprise a composition comprising a non-peptide surfactant.

The following Examples further illustrate the present invention but should not be construed as in any way limiting its scope.

### EXAMPLES

### Example 1

### Designer Short Peptide Surfactants Stabilize G Protein-Coupled Receptor Bovine Rhodopsin

Membrane proteins play vital roles in every aspect of cellular activities. To study diverse membrane proteins, it is crucial to select the right surfactants to stabilize them for analysis. Despite much effort little progress has been made in elucidating their structure and function, largely because of a lack of suitable surfactants. Here we report the stabilization of a G protein-coupled receptor bovine rhodopsin in solution, using a new class of designer short and simple peptide surfactants. These surfactants consist of seven amino acids with a hydrophilic head, aspartic acid or lysine, and a hydrophobic tail with six consecutive alanines. These peptide surfactants not only enhance the stability of bovine rhodopsin in the presence of lipids and the common surfactants n-dodecyl-D-maltoside and octyl-D-glucoside, but they also significantly stabilize rhodopsin under thermal denaturation conditions, even after lipids are removed. These peptide surfactants are simple, versatile, effective, and affordable. They represent a designer molecular nanomaterial for use in studies of diverse elusive membrane proteins.

Membrane proteins are involved in all aspects of vital cellular activities including energy conversion, photosynthetic electron transport, cell signaling, cell-cell interactions, cell adhesion, cell migration and movement, cytoskeletal organization, protein trafficking, viral fusion, information propagation, cellular secretor, and neural synaptic activities, ion and metabolite transport, and respiratory transport. An approximate one-third of the genes in the human genome code for membrane proteins. Of that number, only a single human membrane protein structure (that of monoamine oxidase B) has been determined by x-ray diffraction at 3-Å resolution (1). To a lesser resolution (3.8 Å), the structure of human aquaporin 1 was determined by electron crystallographic diffraction (2). Thus, membrane protein structures largely remain elusive, primarily because of a lack of the right surfactants.

G protein-coupled receptors (GPCRs) comprise a large class of membrane proteins and play a crucial role in the signaling cascade (3, 4). Although they are vitally important in the pharmaceutical and biotechnology industries, medical science, and nanobiotechnology (3, 4), only a single bovine rhodopsin structure is known (5-8). To study the structure and function of diverse membrane proteins, including GPCRs, it is crucial to discover, search, design, synthesize, and select the right surfactants to stabilize membrane proteins.

It is estimated through extensive bioinformatics studies that approximately one-third of the total number of genes in sequenced organisms' genomes code for membrane proteins (9-1). Despite the importance of membrane proteins, dynamic studies of them and methods for their high-resolution structural analysis are rather limited. Although >35,000 soluble protein structures have been elucidated (Protein Data Batik, www.resb.org/pdb), only 215 membrane proteins, including 113 unique structures, have been determined as of July 2006 (http://blanco.biomol.uci.edu/membrane/proteins/xtal.html). Thus, membrane proteins pose a grand challenge that requires new tools, materials, and methods for systematic structural and other studies. Although numerous surfactants are available and have been used for many years in membrane protein studies, none have been completely satisfactory for use in stabilizing diverse membrane proteins, which quickly denature or aggregate in solution. New types of surfactants that can preserve membrane protein stability, structure, and function are prerequisites in tackling the problem and are urgently needed.

Previously, limited numbers of membrane proteins were solubilized, stabilized, and crystallized by using a variety of surfactants (12, 14-17). Most surfactants that have been successfully used contain a hydrophilic head and a hydrophobic tail that consists of 6-12 carbon atoms (12, 18-20). Surfactants with short hydrophobic tails prove to be the most useful for crystallization (21). In addition, several α-helical peptide surfactants and composite α-helix peptides with lipid tails have been reported to stabilize membrane proteins (22-24). However, these α-helical peptides are >30 residues long and expensive to obtain; they also require proper folding before they attain the properties of a surfactant.

We previously reported the design of a class of self-assembling amphiphilic peptide surfactants that include G₄D₂, G₆D₂, G₈D₂, A₆D, A₆K, V₆D, V₆K, and L₆D₂ (25-27). These surfactants comprise ≈6-10 amino acid residues, are ≈2-3 nm in length, and have properties similar to those of common surfactants, such as n-dodecyl-B-D-maltoside (DM) and octyl-D-glucoside (OG). These peptides have one or two hydrophilic amino acids at one end, with either a negatively charged aspartic acid or a positively charged lysine followed by several consecutive hydrophobic amino acids such as glycine, alanine, valine, and leucine. When the N terminus is acetylated, the surfactants A₆D and V₆D) have two negative charges, one front the C terminus and the other from the aspartic acid side chain. On the other hand, A₆K has one negative charge from the C terminus and a positive charge from the lysine side chain. When dissolved in water or ionic solutions, these peptide surfactants undergo self-assembly to form micelles, nanovesicles, or nanotubes (25-27).

Similar to common surfactants, these peptide surfactants have defined critical aggregation concentrations (CACs) in the sub-millimolar to millimolar range, depending on the hydrophobicity of the tails and the ionic concentration. For example, in water, A₆D has a CAC of ≈1.6 mM, and A₆K has a CAC of 1.5 mM; however, in phosphate-based saline (PBS) (10 mM sodium phosphate/150 mM NaCl, pH 7.4), A₆D has a CAC of 0.25 mM and A6K has a CAC of ≈0.23 mM. This difference is due the charge screening effect in accordance with the Derjaguin-Landau-Verwey-Overbeck (DVLO) theory, which postulates that a critical coagulation concentration of counterions is required to allow assembly and that this concentration will be inversely proportional to the valence of the counterion raised to the sixth power (28). The peptide surfactant supramolecular structure is similar to that of phospholipids; namely, the formation of a polar interface sequesters the hydrophobic tails from water.

We asked whether the peptide surfactants are capable of stabilizing a well characterized membrane protein such as GPCR bovine rhodopsin. Rhodopsin consists of seven transmembrane helices that form a binding pocket for the chromophore, 11-cis-retinal (29, 30). In a dark state, rhodopsin has an absorbance maximum of 500 nm. Oil absorption of a photon, the retinal isomerizes from 11-cis to all-trans and, concomitantly, rhodopsin undergoes conformational changes that result in an activated state Meta II, which has a maximum visible absorbance of 380 nm (31-34). Rhodopsin can be thermally denatured, which leads to the loss of the 500-nm absorbing chromophore (A500). Here we report that the peptide surfactants stabilize GPCR bovine rhodopsin more effectively than the other common surfactants that have been tested so far. We also show that the designed peptide surfactants stabilize other membrane proteins, i.e. the photosystem I complex. Our studies suggest that short peptide surfactants may be promising material for further studies of membrane proteins.

### Results

### Peptide Surfactant A6D Enhances the Thermal Stability of GPCR Bovine Rhodopsin.

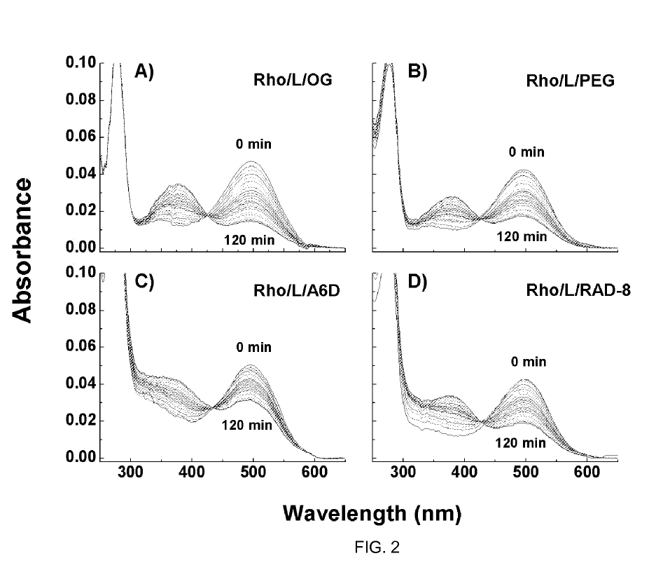

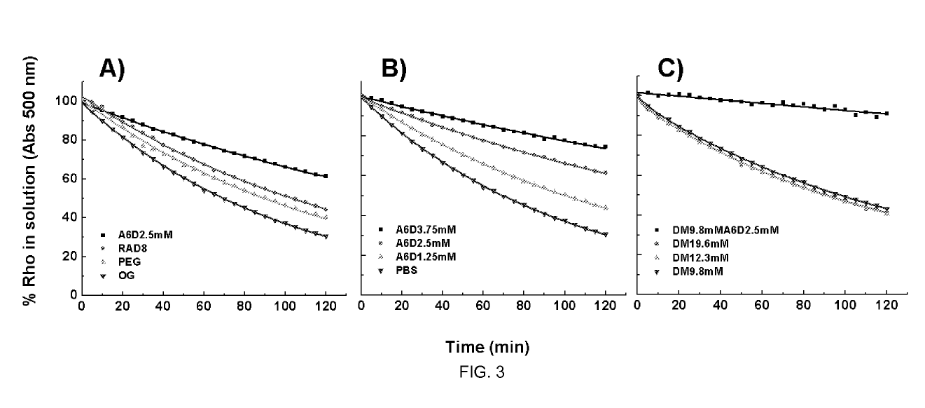

We examined the thermal stability of bovine rhodopsin in the presence of several surfactants. First, we extracted the rhodopsin from bovine retinae rod outer segments by using 2% OG (CAC of ≈5 mM) and obtained rhodopsin in phospholipids/OG mixed micelles. We then measured the thermal stability of rhodopsin in different surfactants and PBS solutions by following the absorbance decrease in A500 as a function of incubation time. We found that rhodopsin in a rhodopsin/phospholipids/OG complex displayed a thermal half-life of ≈71 min (FIGS. 2 and 3 A and B) When 2.5 mM A6D (≈10×CAC in PBS) was added to rhodopsin/phospholipids/OG mixed micelles, the half-life of rhodopsin increased to ≈173 min, representing a 2.5-fold increase in stability compared with the stability in OG alone (FIGS. 2 and 3 A and B). To rule out nonspecific chromophore stabilization, we used another octapeptide-RAD8 (Ac-n-RADARADA-c-NH2), which is similar in size to A₆D without significant surfactant properties. Rhodopsin in phospholipid/OG/RAD8 mix displayed a half-life of ≈99 min (FIGS. 2 and 3A).

It is known that proteins are usually more stable at higher concentrations. To rule out the concentration effect of A6D nonspecifically increasing rhodopsin stability, we added 0.9% PEG 1000 to the rhodopsin/lipid/OG complex. We observed that rhodopsin had a half-life of ≈90.0 min after a 2-h incubation; no significant increase in thermal stability was observed (FIGS. 2 and 3A). These results suggest that enhanced stability is not due to higher concentrations of substance; rather, it is plausible that A₆D formed mixed micelles with OG and phospholipid and effectively enhanced rhodopsin stability against thermal denaturation.

To evaluate the effectiveness of peptide surfactant A₆D in stabilizing rhodopsin's structural integrity in the rhodopsin/phospholipids/OG/A₆D complex, we carried out experiments by using various peptide surfactant A₆D concentrations. We prepared rhodopsin/lipid/OG complex containing different concentrations of A6D, at 1.25 mM (≈5×CAC), 2.5 mM (≈10×CAC), and 3.75 mM (≈15×CAC), and then measured the loss of absorbance at 500 nm as a function of incubation time (FIG. 3B). We found that the stability half-life of rhodopsin increased from ≈71 min, without A₆D, to ≈101, ≈173, and ≈277 min, respectively, as a function of A₆D concentration increase. The nearly 4-fold increase in stability of rhodopsin was observed with the highest A₆D concentration (3.75 mM) (FIG. 3B). These observations suggest that peptide surfactant A₆D effectively stabilized GPCR bovine rhodopsin.

To determine whether the effect of A₆D on stabilizing rhodopsin is specifically associated with OG, we combined A₆D with DM (CAC of ≈0.15 mM) and measured rhodopsin half-life in rhodopsin/lipid/DM preparations. DM is one of the most common surfactants for solubilizing and stabilizing membrane proteins, and it has been shown to stabilize rhodopsin in the absence of lipids at room temperature in ≈2 days at 40° C. We first prepared samples containing different concentrations of DM to determine DM saturation concentration so that we could use peptide surfactant A₆D to test whether the addition of A₆D further enhances rhodopsin stability. We prepared samples containing different DM concentrations. We found that the half-life of rhodopsin in rhodopsin/phospholipids/9.8 mM DM (65×CAC), rhodopsin/phospholipid/13.3 mM DM (89×CAC), or rhodopsin/phospholipids/19.6 mM DM (130×CAC) was ≈102, ≈93 and ≈93 min, respectively.

The results showed that increasing DM concentration atone is insufficient to enhance bovine rhodopsin thermal stability. At a defined DM saturation concentration, we then added A6D to rhodopsin prepared in the DM concentration given above and monitored its thermal stability. Our results showed that, when the rhodopsin/phospholipids/DM sample was supplemented with an additional 2.5 mM A₆D (10×CAC), the rhodopsin became extremely stable with little decay, even at 50° C. (FIG. 3C). These results demonstrate that A₆D significantly enhances rhodopsin stability against thermal denaturation when A₆D and DM are combined to elevate surfactant effectiveness in protecting rhodopsin stability, possibly in a synergistic way.

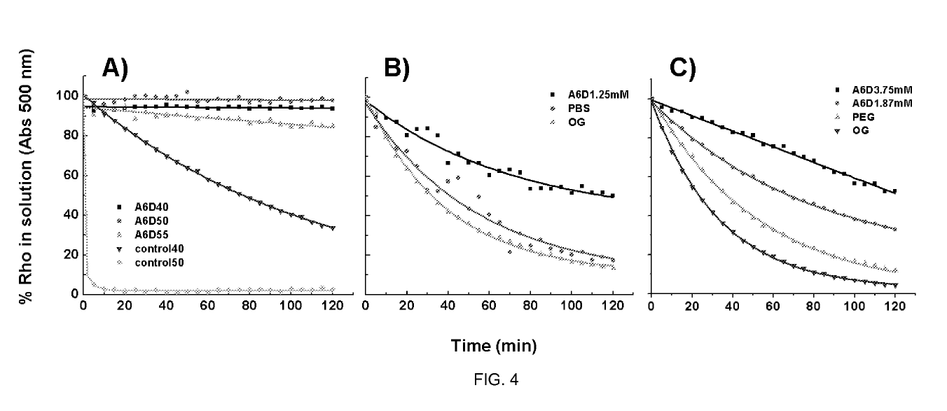

Peptide Surfactant A6D Stabilized Rhodopsin in the Absence of OG. We then asked whether the peptide surfactant A₆D alone, without 0 or DM, could stabilize rhodopsin. We then removed OG from rhodopsin/lipid/OG mixture through extensive dialysis against buffers containing insignificant 0 (defined as no OG and then monitored rhodopsin thermal stability at 40° C., 50° C., and 55° C. Surprisingly, the rhodopsin/lipid/A₆D complex without OG was very stable at all tested temperatures with little denaturation. In contrast, rhodopsin in control samples containing only OG, without A₆D, had a half-life of ≈101 min at 40° C. and ≈5 min at 50° C. (FIG. 4A). These results suggest not only that A6D alone is sufficient to stabilize rhodopsin but also that peptide surfactant A₆D can be used for protecting rhodopsin in solution without the common surfactants OG and DM. This observation opens the wary to test the ability of A₆D to stabilize and crystallize membrane proteins.

Peptide Surfactant A6D Stabilized Rhodopsin in the Absence of Lipid and OG. To determine whether A₆D can substitute for phospholipids, we carried out, experiments to test rhodopsin stability as a function of A6D concentration. We prepared four delipidated rhodopsin samples in (i) OG, (ii) 0.9 PEG in OG, (iii), 1.87 mM A₆D in OG, and (iv) 3.75 mM A₆D in OG. We then measured the thermal stability of these samples at 40° C. The delipidated rhodopsin in the OG/PEG mixture without A₆D had the shortest half-life, ≈27 min. The stability of rhodopsin in A₆D was increased as a function of the increasing concentration of A6D (FIG. 4B). Rhodopsin samples containing A₆D at 1.87 mM (≈7×CAC) or 3.75 mM (≈15×CAC) displayed a half-life of ≈76 and ≈128 min, respectively. This result represents a 2.8- to 4.7-fold increase in stability compared with the stability in OG alone (FIG. 4B). These results suggest that A₆D stabilizes rhodopsin effectively, even after lipid removal.

We then asked whether removing residue phospholipids from the preparation, namely, A₆D alone without endogenous residue phospholipids, could stabilize delipidated rhodopsin. We carried out experiments to purify delipidated rhodopsin, using anti-rhodopsin-1D4 Ab immunoaffinity chromatography to remove the endogenous phospholipids from the rhodopsin preparation by extensive washing with the peptide surfactant A₆D and replacing the phospholipids with A₆D. This method has been successfully used to remove phospholipids from rhodopsin purified from bovine retinae rod outer segments. After affinity purification to obtain delipidated rhodopsin, we tested rhodopsin stability in the absence of both OG and phospholipids. We found that delipidated rhodopsin in the absence of both phospholipids and OG had a short half-life of ≈47 min at 40° C. Furthermore, adding OG to the delipidated rhodopsin did not have a significant stabilization effect. On the other hand, delipidated rhodopsin stabilized by A6D alone had a half-life of ≈122 min at 40° C., a 2.6-fold increase in stability (FIG. 4C).

### DISCUSSION

Surfactants play a vital role in our understanding of the structure and function of membrane proteins. Numerous attempts halve been made to discover, synthesize, and select a variety of surfactants to facilitate the study of membrane proteins, but little progress has been made so far. It is widely known that obtaining stable membrane proteins for high-resolution structures requires the right surfactants. The designer short peptide surfactants reported here belong to a class of the simplest surfactants, which are easily adapted for molecular engineering for individual membrane proteins.

We have found that this class of designed peptide surfactants can stabilize not only the function of GPCR bovine rhodopsin in solution but also multiple subunits of the photosystem I (PSI) protein complex (35) on a dry surface (36, 37). These peptide surfactants represent a promising approach to membrane protein crystallization. V₆D was used to solubilize the integral membrane protein glycerol 3-phosphate dehydrogenase in solution, when assayed through protein gel electrophoresis, retaining its functional enzymatic activities (338).

This class of short peptide surfactants, including A₆D, V₆D, A₆K, and others, can be very useful. We show here that A₆D not only can synergistically interact with the common surfactants OG and DM and lipids to enhance rhodopsin stability, but it also can effectively protect rhodopsin function against thermal denaturation in the absence of both lipid and common surfactants. This observation suggests that peptide surfactants interact with membrane proteins to stabilize them. Indeed, our previous results showed that peptide surfactants undergo self-assembly to form micelles, nanovesicles, and nanotubes (25-27).

These short peptide surfactants may have several advantages in studying diverse membrane proteins. (i) Their biochemical properties resemble common surfactants with similar CACs and seem not to denature membrane proteins and membrane protein complexes, as shown by our studies and those of other researchers. (ii) They are chemically and structurally simple and can be adapted quickly, for a wide variety of uses. (iii) They are short with high purity, soluble in water, and stable for long periods at ambient temperature. (iv) they are affordable worldwide because peptide manufacturing is a mature industry, and the price is decreasing steadily. (v) They can readily be used with other common surfactants in a combinatorial manner.

Short Peptide Surfactants Stabilize Other Membrane Proteins. We used green plant PSI (35) to demonstrate that these designer short peptide surfactants can stabilize membrane proteins (36, 37). PSI is a chlorophyll-containing membrane protein complex that is the primary reducer of ferredoxin and the electron acceptor of plastocyanin. We isolated the complex from the thylakoids of chloroplasts by using a common surfactant, Triton X-100. The chlorophyll molecules associated with the PSI complex provide an intrinsic steady-state emission spectrum between 650 and 800 nm at 77 K that reflects the organization of the pigment-protein interactions. In the absence of surfactants, a large blue shift of the fluorescence maxima from ≈735 nm to ≈685 nm indicates a disruption in light harvesting subunit organization, thus disrupting chlorophyll-protein interactions. The commonly used membrane protein-stabilizing surfactants, DM and OG, did not stabilize the ≈735-nm complex with the ≈685-nm spectroscopic shift. However, before drying the sample, addition of the peptide surfactant Ac-AAAAAAK (A6K) at an increasing concentration significantly stabilized the PSI complex (336). Moreover, in the presence of the A6K peptide surfactant, the PSI complex is stable in a dried form at room temperature for at least 3 weeks (36, 37). Another peptide surfactant, Ac—VVVVVVD (V₆D), also stabilized the complex, but to a lesser extent. These observations suggest that peptide surfactants may stabilize membrane protein complexes on a dry surface.

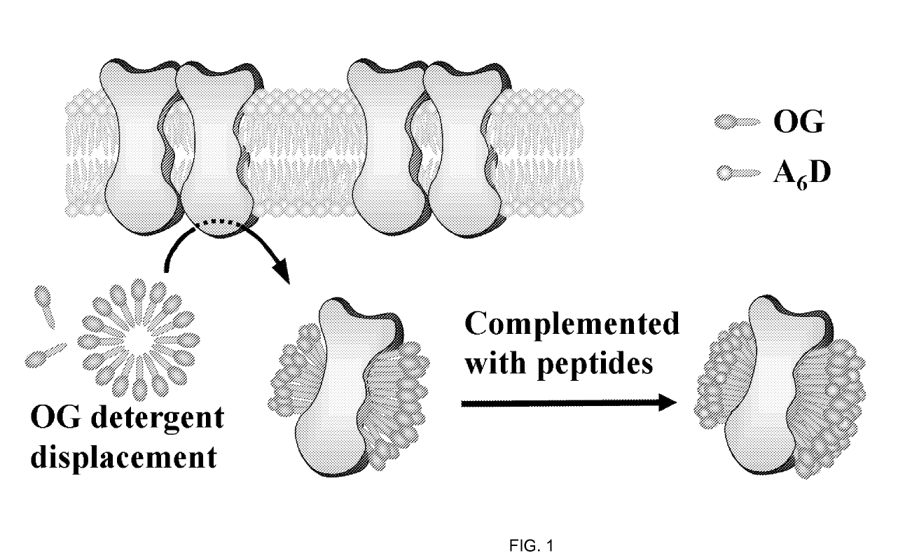

Proposed Model of How Peptide Surfactants Interact with Membrane Proteins. We now wish to introduce a plausible model to explain how simple peptide surfactants interact with membrane proteins, particularly rhodopsin or other GPCRs (FIG. 1). In this model, the peptide surfactants form micelles and other nanostructures in the absence of proteins. When membrane proteins, e.g., rhodopsin, are present, these small peptide surfactants surround rhodopsin and act to protect it from thermal denaturation, similar to the action of chaperones, lipids, and other surfactants.

Perspective on Designer Lipid-Like Peptides for Membrane Protein Studies. The field of designer short and simple peptide surfactants is in its infancy. However, several observations described here indicate that this class of surfactants will be very useful. We have designed a small number of variations from 2.0 natural L-amino acids, not all in mirror image, of 20 D-amino acids, as well as an increasing number of unnatural amino acids. All of these amino acids can be incorporated into the class of short peptide surfactants. A combinatorial approach can be readily applied to produce peptides with a wide range of properties for membrane protein study. So far, we have only focused on peptide surfactants containing homogenous hydrophobic tails. One possibility for the specialization of molecules is the exploration of mixtures of short and long tails; heterogeneous tails; tails with many hydrophobic residues including valine, leucine, methanine, isoleucine, proline, phenylalanine, tyrosine, tryptophan; and different head groups including sugars and other polar molecules. Peptides that further enhance stabilization and crystallization of membrane proteins might then be identified. Another possibility is the exploration of mixing several different surfactants together as cocktails. Naturally, proteins can select the most appropriate peptide components from such a mixture for use in stabilization and crystallization to uncover their structure and function,

### Methods

GPCR Bovine Rhodopsin Source. Frozen bovine retinae were obtained from J. A. Lawson (Lincoln, Nebr.). Cell culture media and supplements were from Irvine Scientific (Santa Ana, Calif.) and Sigma (St. Louis, Minn.).

Chemicals, Surfactants, and Peptide Surfactants. DM was purchased from Anatrace (Maumee, Ohio). OG was purchased from Roche Molecular Biochemicals (Mannheim, Germany). CNBr-activated Sepharose was purchased from Amersham Pharmacia (Little Chalfont, U.K.). All designed peptide surfactants were customary synthesized and characterized by the Biopolymers Laboratory at the Massachusetts Institute of Technology and Synpep (Dublin, Calif.).

Purification Materials. Peptides corresponding to the C terminus (T340-A348) of rhodopsin were synthesized by the Biopolymers Laboratory at the Massachusetts Institute of Technology and purified by HPLC. Rhodopsin nonapeptide T340-A348 was used to elute rhodopsin from rhodopsin-1D4 Sepharose beads at a concentration of 100 uM throughout.

mAbs Coupled to Sepharose Beads. Anti-rhodopsin mAb rhodopsin-1D4 (39) was prepared by the National Cell Culture Center (Minneapolis, Minn.). The mAb rhodopsin-1D4 was coupled to CNBr-activated Sepharose beads as described (40, 41), except that 10 mg of the purified mAb proteins was bound per 1 ml of rehydrated beads. The resulting mAb rhodopsin-1D4 Sepharose beads had a capacity to bind 1 mg of rhodopsin per milliliter of settled beads.

Solubilization of Rhodopsin in Different Surfactants. Bovine retinae rod outer segment membranes were prepared (40, 41) and urea-stripped (42). Samples of the membranes containing 1.4 nmol of rhodopsin were solubilized in 0.5 ml of PBS containing 0.5 mM phenylmethylsulfonyl fluoride with 1% DM or 2% (/vol) OG. The suspensions were centrifuged (100,000×g for 15 min), and the supernatants were kept in the dark at 4° C.

Thermal Stability Study of Rhodopsin in Peptide Solution in the Presence of the Surfactant. Rhodopsin solubilized (32-34) in PBS containing 2% (vol/vol) OG was added to the same volume of PBS containing 0, 25, 5.0, or 7.5 mM A6D, 1.8% PEG, or 5.4 mM RAD8. In another experiment, rhodopsin solubilized in PBS containing 1% (19.8 mM) DM was added to the same volume of PBS containing 5.0 mM A6D or 0, 5.0, or 19.8 mM DM. A decrease in A500 was recorded every 5 min up to 110 min at 40° C. for the samples containing OG and at 40-55° C. for those containing DM (FIGS. 2 and 3). UV-visible absorption spectroscopy was performed with a Lambda 6 spectrophotometer (Perkin-Elmer, Wellesley, Mass.) equipped with a temperature regulated cuvette holder with a slit width of 2 nm, 480 nm/min scan speed, and a response time of 1 second. The A500 remaining at different time points was expressed as the percentage of absorbance present at the start of the experiment. The half-life of rhodopsin in each solution was calculated by using a single exponential curve fit. The plot obtained from the percentage of absorbance at different time points was fitted to a single exponential curve with two parameters (Eq. 1).

y=a exp(−bx) [Eq. 1]

The plot obtained from the A500 values at different time points was directly fitted to a single exponential curve with three parameters (Eq. 2) when the plot from the percentage of absorbance could not be fitted to Eq. 1.

y=y0+a exp(−bx) [Eq. 2]

The half-life (t_{1/2}) was calculated using Eq. 3.

t_{1/2}=ln(2)/b [Eq. 3]

Thermal Stability of Rhodopsin in Peptide Solution in the Absence of OG. Rhodopsin solubilized in PBS containing 2% (vol/vol) OG was added to the same volume of PBS containing 2.5 mM A6D. Then, 0.5 ml of the mixture was poured into the tube 2.5 mM A6D. Then, 0.5 ml of the mixture was poured into the tube. The tube was covered with 10 kDa of cutoff membrane and dialyzed against 4.5 ml of PBS containing 1.25 mM A6D for 40 h in the dark at 4° C. A resultant sample decrease in A500 was recorded every 5 min for 120 min at 40° C. A decrease in A500 of the predialysis mixture containing 1% OG and 1.25 mM A6D was also monitored as a control (FIG. 4A).

Removal of Phospholipids from Rhodopsin. Samples of the membranes containing 4.7 nmol of rhodopsin were solubilized in 1,000 ul of PBS containing 2% (vol/vol) OG. The suspension was mixed with 250 ul (settled beads, concentration of 50%) of rhodopsin-1D4 Sepharose and rotated for 3 h. The 1D4 Sepharose beads were then packed into a minicolumn (7-mm inner diameter) and washed with PBS containing 1% OG by using 500 bed volumes (total of 62.5 ml). The rhodopsin was then eluted with 1.5 ml of PBS containing 1% OG and the C-terminal nanopeptide.

Thermal Stability Study of Rhodopsin in Peptide Solution in the Absence of Phospholipids. The delipidated rhodopsin solution was added to the same volume of 1% OG in PBS containing 0, 3.75, or 7.5 mM A6D, or 1.8% PEG (FIG. 4B). In another experiment, the delipidated rhodopsin solutions were poured into tubes. Each tube was covered with 10 kDa of cutoff membrane and dialyzed against 4.5 ml of PBS or PBS containing 1.25 mM A6D or 1% OG (FIG. 4C). For all samples, a decrease in A500 was recorded every 5 min, up to 120 min at 40° C.

### Example 2

### Stabilization of the Large Membrane-Associated Protein Complex, Using Lipid-Like Peptide Surfactants

Introduction: The stabilization of large protein complexes in a functional manner is of fundamental importance to many technological applications including biosensors, bioactive tissue scaffolds. Of particular interest is the stabilization of large membrane bound and membrane associated, protein complexes as they present unique functionalities and could be the basis for novel sensing technologies. The control over the backbone structure of lipid-like peptide surfactants offers a powerful tool for the design of protein stabilizing compounds. Recent work has shown that lipid-like peptides are able to stabilize complex membrane proteins [43]. However, it is currently unknown if this strategy can be employed for stabilizing membrane associated super-complexes (˜10 Million Daltons), composed of several hundred proteins that self-assemble into a highly ordered 3-D structure that is required for function.

For this purpose, a model protein complex, phycobilisomes from cyanobacteria, was selected for stabilization studies. Phycobilisomes are photo-antennae that are associated with the membrane bound reaction center of cyanobacteria. This protein complex presents a unique structure that can adsorb multiple photon energies (450-600 nm), transporting this energy to the protein core, where the energy is fluoresced at a single wavelength (666 nm). It is well known that the energy transport properties of phycobilisomes are severely affected when slightly denatured. Therefore, these unique properties make Phycobilisomes an ideal protein complex to determine the stabilization efficacy of peptergents using simple optical techniques, like fluorescent spectroscopy.

Methods: Native phycobilisomes were isolated from Porphridium Cruentum, purified by Martek Biosciences and received in stabilizing buffer (0.75 M PBS, pH ˜7). Peptergents (ac-A6D and ac-A6K—NH₂) were used as supplied (CPC Scientific) without further purification. Briefly, phycobilisomes (1 ng/mL) were mixed with peptergent-0.75M PBS solutions (0, 1, 2, 4 and 8 critical aggregation concentrations (CAC)). Phycobilisome-peptide solutions were dialyzed (3400 MWCO) against PBS solutions of various molarity (0.6-0.01M) for 18 h, in the dark. Room temperature fluorescent spectroscopy of these solutions was conducted using an excitation wavelength of 540 nm and emitted fluorescence (550-700 nm) were collected. UV-Vis spectra were obtained to confirm solution concentrations for all acquired fluorescent data.

Results/Discussion: It is evident that upon changing the buffer to 0.4M PBS, the intensity of the 666 nm fluorescent peak decreases to background. UV-Vis data illustrate that the concentration of phycobilisome in the unstable control is only 15% less than that of the stabilized control; thus, the lack of 666 nm signal is not due to the lack of phycobilisome but rather due to protein denaturation. Finally, it is evident that upon addition of both A6D and A6K the phycobilisome maintains its functional structure, yielding 666 nm intensities of 1.3 and 0.7×10⁵ cps. Both A6D and A6K solutions were similar in phycobilisome concentration, both being ˜15% lower than the concentration of the stabilized control solution. Concentration normalized signal strength for A6D stabilized phycobilisomes is very similar to the stabilized control. However, the presence of a new peak at ˜600 nm may indicate a slight perturbation of the phycobilisome structure that is preventing optimal energy transfer to the core of the protein complex.

These results show that peptergents are powerful molecules that are able to stabilize large, membrane associated, protein complexes, representing significant progress in the development of these technologies for protein-based biotechnological devices.

### REFERENCES

1. Binda C, Newton-Vinson P, Hubalek F, Edmondson D E, Mattevi A (2002) Nat Struct Biol 9:922-926.
2. Murata K, Mitsuoka K, Hirai T, Walz T, Agre P, Heymann J B, Engel A, Fujiyoshi Y (2000) Nature 407:599-605.
3. Watson S, Arkinstall S (1994) The G-Protein Linked Receptor Facts Book (Academic, London).
4. Haga T, Berstein G (1999) G Protein-Coupled Receptors (CRC, Boca Raton, Fla.).
5. Palczewski K, Kumasaka T, Hori T, Behnke C A, Motoshima H, Fox B A, Le Trong I, Teller D C, Okada T, Stenkamp R E, et al. (2000) Science 289: 739-745.
6. Okada T, Fujiyoshi Y, Silow M, Navarro J, Landau E M, Shichida Y (2002) Proc Natl Acad Sci USA 99:5982-5987.
7. Okada T, Sugihara M, Bondar A N, Elstner M, Entel P, Buss V (2004) J Mol Biol 342:571-583.
8. Li J, Edwards P C, Burghammer M, VIIIa C, Schertler G F (2004) J Mol Biol 343:1409-1438.
9. Wallin E, von Heijne G (1998) Protein Sci 7:1029-1038.
10. Kall L, Sonnhammer E L (2002) FEBS Lett 532:415-418.
11. Liu Y, Engelman D M, Gerstein M (Sep. 19, 2002) Genome Biol, 10.1186_gb-2002-3-10-research0054.
12. Loll P J (2003) J Struct Biol 142:144-153.
13. Hessa T, Kim H, Bihlmaier K, Lundin C, Boekel J, Andersson H, Nilsson I, White S H, von Heijne G (2005) Nature 433:377-381.
14. Gennis R B (1989) Biomembranes: Molecular Structure and Function (Springer, New York).
15. Michel H (1990) Crystallization of Membrane Proteins (CRC Press, Boca Raton, Fla.).
16. Ostermeier C, Michel H (1997) Curr Opin Struct Biol 7:697-701.
17. le Maire M, Champeil P, Moller J V (2000) Biochim Biophys Acta 1508:86-111.
18. Ringler P, Heymann B, Engel A (2000) in Membrane Transport, Practical Approach Series, ed Baldwin S A (Oxford Univ Press, Oxford), pp 229-268.
19. Howard T A, McAuley-Hecht K E, Cogdell R J (2000) in Membrane Transport, Practical Approach Series, ed Baldwin S A (Oxford Univ Press, Oxford), pp 269-307.
20. Garavito R M, Ferguson-Miller S (2001) J Biol Chem 276:32403-32406.
21. Hauser H (2000) Biochim Biophys Acta 1508:164-181.
22. Schafmeister C E, Miercke L J, Stroud R M (1993) Science 262:734-738.
23. McGregor C L, Chen L, Pomroy N C, Hwang P, Go S, Chakrabartty A, PriveGG (2003) Nat Biotechnol 21: 171-176.
24. Soomets U, Kairane C, Zilmer M, Langel U (1997) Acta Chem Scand 51(3 Suppl):403-406.
25. Vauthey S, Santoso S, Gong H, Watson N, Zhang S (2002) Proc Natl Acad Sci USA 99:5355-5360.
26. Santoso S, Hwang W, Hartman H, Zhang S (2002) Nano Lett 2:687-791.
27. von Maltzahn G, Vauthey S, Santoso S, Zhang S (2003) Langmuir 19:4332-4337.
28. Verwey E J W, Overbeek J T G (1948) Theory of the Stability of Lyophobic Colloids (Elsevier, Amsterdam) pp 106-115.
29. Gerber G E, Gray C P, Wildenauer D, Khorana HG (1977) Proc Natl Acad Sci USA 74:5426-5430.
30. Mogi T, Stern L J, Marti T, Chao B H, Khorana H G (1988) Proc Natl Acad Sci USA 85:4148-4152.
31. Grobner G, Burnett I J, Glaubitz C, Choi G, Mason A J, Watts A (2000) Nature 405:810-813.
32. Resek J F, Farahbakhsh Z T, Hubbell W L, Khorana H G (1993) Biochemistry 32: 12025-12032.
33. Reeves P J, Thurmond R L, Khorana H G (1996) Proc Natl Acad Sci USA 93:11487-11492.
34. Reeves P J, Callewaert N, Contreras R, Khorana H G (2002) Proc Natl Acad Sci USA 99:13419-13424.
35. Ben-Shem A, Frolow F, Nelson N (2003) Nature 426:630-635.
36. Das R, Kiley P J, Segal M, Norville J, Yu A, Wang L, Trammell S, Reddick L E, Kumar R, Stellacci F, et al. (2004) Nano Lett 4:1079-1083.
37. Kiley P, Zhao X, Bruce B D, Baldo M, Zhang S (2005) PLoS Biol 3:1180-1186.
38. Yeh J I, Du S, Tordajada A, Paulo J, Zhang S (2005) Biochemistry 44:16912-16919.
39. Oprian D D, Molday R S, Kaufman R J, Khorana H G (1987) Proc Natl Acad Sci USA 84:8874-8878.
40. Papermaster D S (1982) Methods Enzymol 81:48-52.
41. Papermaster D S (1982) Methods Enzymol 81:240-246.
42. Shichi H, Somers R L (1978) J Biol Chem 253:7040-7046.
43. Kiley, P. et al., PloS Biology. 2005; 3, 1181-1186.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of stabilizing a mammalian membrane protein comprising adding a surfactant peptide to the mammalian membrane protein, wherein the surfactant peptide has a formula selected from the group consisting of:
a. (Φ)ₘ(+)ₙ (Formula (I)),
b. (+)ₙ(Φ)ₘ (Formula (II)),
c. (Φ)ₘ(−)ₙ (Formula (III)),
d. (−)ₙ(Φ)ₘ (Formula (IV)),
e. (−)ₙ(Φ)ₘ(−)ₙ (Formula (V)),
f. (+)ₙ(Φ)ₘ(+)ₙ (Formula (VI)),
g. (Φ)ₘ(−)ₙ(Φ)ₘ (Formula (VII)),
h. (Φ)ₘ(+)ₙ(Φ)ₘ (Formula (VIII)),
i. (+)ₙ(Φ)ₘ(−)ₙ (Formula (IX)),
j. (−)ₙ(Φ)ₘ(+)ₙ (Formula (X)),
k. (τ)ₙ(Φ)ₘ (Formula (XI)),
l. (Φ)ₘ(τ)ₙ (Formula (XII)),
m. (τ)ₙ(Φ)ₘ(τ)ₙ (Formula (XIII)), and
n. (Φ)ₘ(τ)ₙ(Φ)ₚ (Formula (XIV)),
wherein:
the last amino acid in the peptide sequence is the C-terminus;
each (Φ) is independently a non-polar amino acid containing a non-charged side chain at physiological pH;
each (+) is independently an amino acid containing a cationic side chain at physiological pH;
each (−) is independently an amino acid containing an anionic side chain at physiological pH;
each (τ) is independently is a polar amino acid containing a non-charged side chain at physiological pH, for example, serine, threonine, asparagine and glutamine;
each m is independently an integer greater than or equal to 4; and
each n is independently an integer greater than or equal to 1.

2. The method of claim 1, wherein each (Φ) is independently selected from the group consisting of glycine, alanine, valine, leucine and isoleucine, methionine, phenylalanine, tyrosine and tryptophan.

3. The method of claim 1, wherein each (+) is independently selected from the group consisting of lysine and arginine.

4. The method of claim 1, wherein each (−) is independently selected from the group consisting of aspartic acid and glutamic acid.

5. The method of claim 1, wherein each (τ) is independently selected from the group consisting of serine and threonine.

6. The method of claim 1, wherein the surfactant peptide has the formula (Φ)ₘ(+)ₙ.

7. The method of claim 5, wherein the surfactant peptide is selected from the group consisting of Ac-AAAAAAK—NH₂ (A₆K), Ac—VVVVVVK—NH₂ (V₆K), Ac—IIIIK—NH₂ (I₄K), Ac—IIIIIIKK—NH₂ (T₆K₂), Ac—VVVVVVKK—NH₂ (V₆K₂) and Ac—VVVVVVRR—NH₂ (V₆R₂).

8. The method of claim 1, wherein the surfactant peptide has the formula (+)ₙ(Φ)ₘ.

9. The method of claim 7, wherein the surfactant peptide is selected from the group consisting of KKLLLLLL-NH₂ (K₂L₆), KKVVVVVV—NH₂ (K₂V₆) and KAAAAAA-NH₂ (KA₆).

10. The method of claim 1, wherein the surfactant peptide has the formula (Φ)ₘ(−)ₙ.

11. The method of claim 9, wherein the surfactant peptide is selected from the group consisting of Ac-AAAAAAD (A₆D), Ac—VVVVVVD (V₆D), Ac—VVVVVVDD (V₆D₂), Ac-LLLLLLD (L₆D), Ac-LLLLLLDD (L₆D₂), Ac—IIIIIID (T₆DD), Ac—FFFFFFD (F₆DD), Ac-GGGGGGD (G₆D), Ac-GGGGGGDD (G₆D₂) and Ac-GGGGGGGGDD (G₈D₂).

12. The method of claim 1, wherein the surfactant peptide has the formula (−)ₙ(Φ)ₘ.

13. The method of claim 11, wherein the surfactant peptide is selected from the group consisting of DIIII—NH₂ (DI₄), DGGGGGG-NH₂ (DG₆), DAAAAAA-NH₂ (DA₆), DDVVVVVV—NH₂ (DV₆), DDIIIIII—NH₂ (DI₆), DDLLLLLL-NH₂ (DL₆) and DDFFFFFF—NH₂ (DF₆).

14. The method of claim 1, wherein the length of the surfactant peptide is from about 2 to about 3 nm.

15. The method of claim 1, wherein the N-terminus of the surfactant peptide is acetylated or methylated.

16. The method of claim 1, wherein the C-terminus is amidated or esterified, e.g., —O—CH₃.

17. The method of claim 14, wherein the surfactant peptide is selected from the group consisting of ac-I₆K₂—CONH₂, ac-A₆K—CONH₂, ac-V₆K₂—CONH₂ and ac-V₆R₂—CONH₂.

18. The method of claim 1, wherein the mammalian membrane protein is a G protein-coupled receptor.

19. The method of claim 1, wherein the mammalian membrane protein is selected from the group consisting of rhodopsin, chemokine (C—C motif) receptor 3 (CCR3), chemokine (C—C motif) receptor 5 (CCR5) and CXCR4 receptor and the list (Table 1).

20. The method of claim 1, wherein at least two surfactant peptides are added to the mammalian membrane protein.

21. The method of claim 1 further adding a non-peptide surfactant is added to the mammalian membrane protein.

22. The method of claim 20, wherein the non-ionic surfactant is selected from the group consisting of n-dodecyl-B-D-maltoside and octyl-D-glucoside.

23. The method of claim 1, wherein the mammalian membrane protein is in an aqueous solution.

24. The method of claim 1, wherein the mammalian membrane protein is in an ionic aqueous solution.

25. The method of claim 1, wherein the mammalian membrane protein is on a dry surface.

26. A kit for stabilizing a mammalian membrane protein comprising a composition comprising a mammalian membrane protein and a composition comprising a surfactant peptide in an amount sufficient to stabilize the mammalian membrane protein, wherein the surfactant peptide is selected from the group consisting of:
a. (Φ)ₘ(+)ₙ,
b. (+)ₙ(Φ)ₘ,
c. (Φ)ₘ(−)ₙ,
d. (−)ₙ(Φ)ₘ,
e. (−)ₙ(Φ)ₘ(−)ₙ,
f. (+)ₙ(Φ)ₘ(+)ₙ,
g. (Φ)ₘ(−)ₙ(Φ)ₘ,
h. (Φ)ₘ(+)ₙ(Φ)ₘ,
i. (+)ₙ(Φ)ₘ(−)ₙ
j. (−)ₙ(Φ)ₘ(+)ₙ,
k. (τ)ₙ(Φ)ₘ,
l. (Φ)ₘ(τ)ₙ,
m. (τ)ₙ(Φ)ₘ(τ)ₙ,
n. (Φ)ₘ(τ)ₙ(Φ)ₚ,
wherein:
the last amino acid in the peptide sequence is the C-terminus;
each (Φ) is independently an amino acid containing a non-charged side chain at physiological pH;
each (+) is independently an amino acid containing a cationic side chain at physiological pH;
each (−) is independently an amino acid containing an anionic side chain at physiological pH;
each (τ) is independently an amino acid containing a uncharged polar side chain at physiological pH, e.g. Ser, Thr, Asn, Gln;
each m is independently an integer greater than 4; and
each n is independently an integer greater than or equal to 1.

27. The kit of claim 24, wherein the kit comprises at least two peptide surfactants.

28. The kit of claim 25, wherein the kit comprises a non-peptide surfactant.

29. The kit of claim 26, wherein the non-peptide surfactant is selected from the group consisting of ñ-dodecy\tilde{l}-B-D-maltoside and octyl-D-glucoside.
